(19) United States
(12) Patent Application Publication
(10) Pub. No.: US 2019/0025234 A1
(43) Pub. Date: Jan. 24, 2019

(54) DETERMINING SOLIDS CONTENT USING DIELECTRIC PROPERTIES

(71) Applicant: Halliburton Energy Services, Inc., Houston, TX (US)

(72) Inventors: Glenn Howard Weightman, Duncan, OK (US); Bruce Carl Lucas, Duncan, OK (US)

(21) Appl. No.: 16/066,889

(22) PCT Filed: Mar. 14, 2016
(86) PCT No.: PCT/US2016/022285

§ 371 Date: Jun. 28, 2018

(51) Int. Cl. G01N 27/22; G01N 33/28; G01N 33/38; E21B 49/08; G01R 27/26

(52) U.S. Cl. CPC G01N 27/221; G01N 33/2823; E21B 2049/085; E21B 49/08; G01R 27/2676; G01N 33/383

(57) ABSTRACT

A method for determining the composition of a two-phase (solid-liquid) slurry that includes determining the dielectric constant of the slurry at various known compositions, correlating the dielectric constant of the slurry with the known compositions and then determining the dielectric constant of an unknown slurry composition and calculating the composition of the slurry based on the relationship between the dielectric constant and the composition of the slurry.

### CROSS-REFERENCE TO RELATED APPLICATIONS

None.

### FIELD

The present invention generally relates to methods for characterizing fluid compositions, particularly determining solids content in a fluid stream. Examples include wellbore servicing fracturing fluids containing proppant material, solids content in a cement slurry and coal content in a coal slurry to name a few.

### BACKGROUND

Hydraulic fracturing is a process commonly used to increase the flow of fluids, such as oil and gas, from a portion of a subterranean formation. Hydraulic fracturing operations generally involve placing a fracturing fluid into a formation or zone at a rate and pressure sufficient to cause the formation to break and form one or more fractures. The fracturing fluids provide two functions, the first is to provide the pressure needed to fracture the formation and the second is to transport solid particles into the fracture to keep the fracture open once the pressure is released and the overburden is permitted to settle. The solid particles, known as proppant or propping agents, can be of various types, such as graded sand, bauxite, ceramics, etc., which are suspended in the fracturing fluid and then deposited in the fractures. By keeping the fracture from fully closing, the proppant particulates aid in forming conductive paths through which fluids may flow. The degree of success of a fracturing operation depends, at least in part, upon fracture conductivity once the fracturing operation has ceased and production has commenced. The fracture conductivity depends, at least in part, on the solids content of the fracturing fluid and the consistency of the mixture of fracturing fluid and proppant. Controlling the solids content of the fluid during fracturing operations can be critical to the operations success, but measuring the solids content of the two-phase mixture can be difficult.

Slurries such as hydraulic cement compositions are commonly employed in the drilling, completion and repair of oil and gas wells. For example, hydraulic cement compositions are utilized in primary cementing operations whereby strings of pipe such as casing are cemented into wellbores. In performing primary cementing, a hydraulic cement composition is pumped into the annular space between the walls of a wellbore and the exterior surfaces of the casing. The cement composition is allowed to set in the annular space, thus forming an annular sheath of hardened substantially impermeable cement. This cement sheath physically supports and positions the casing relative to the walls of the wellbore and bonds the exterior surfaces of the casing string to the walls of the wellbore. The cement sheath prevents the unwanted migration of fluids between zones or formations penetrated by the wellbore. Hydraulic cement compositions are also commonly used to plug lost circulation and other undesirable fluid inflow and outflow zones in wells, to plug cracks and holes in pipe strings cemented therein and to accomplish other required remedial well operations. The integrity of the cementing operation depends, at least in part, on the solids content of the cement composition and the consistency of the mixture. Controlling the solids content of the composition during cementing operations can be critical to the operations success. While cement density is a key parameter in nearly all cement treatments, certain treatments may contain solids additives, e.g. glass beads, which have nearly the same density as the mix water, thus just measuring density may not be a good indicator of the quality of the slurry.

Many other examples exist where determining the solids content in a two-phase flow is desired, they can include: determining the quantity of sand produced with a flowing oil or gas producing stream, determining how much coal is in a coal slurry, determining the solids content in a waste processing stream, among others.

Thus, a need exists for a practical method of determining the solids content of fluids, such as fracturing fluids containing proppant material and cement slurries.

### BRIEF DESCRIPTION OF DRAWINGS

FIG. 1A is a graph of Dielectric Constant versus Water Content.

FIG. 1B is a graph of Dielectric Constant versus Water Content with various known solids concentrations also shown.

FIG. 1C is a graph of Dielectric Constant versus Solids Content.

FIG. 2 shows examples of TDR (Time Domain Reflectometry) soil moisture measurement devices.

FIG. 3 is an illustrative embodiment of a Guided-Wave Radar level sensor.

FIG. 4 is an illustrative embodiment of typical probe installations.

FIG. 5 is an illustrative embodiment of a probe installation parallel to a flow stream.

FIG. 6 is an illustrative embodiment of a probe installation perpendicular to a flow stream.

FIG. 7 is an illustrative embodiment of a probe with protective tube installation parallel to a flow stream.

FIG. 8 is an illustrative embodiment of a probe with protective tube installation perpendicular to a flow stream.

FIG. 9 is an illustrative diagram of a coaxial capacitor.

### DETAILED DESCRIPTION

Disclosed herein are methods of determining the composition of a slurry mixture. An embodiment is a method of determining the solids content in a fluid, such as wellbore servicing fluids, for example a fracturing fluid containing proppant material used to stimulate a subterranean formation.

There can be several embodiments to the present invention, each of which rely on the ability to determine the dielectric constant, εᵣ that can also be referred to as relative permittivity, of a two-phase (solid-liquid) slurry. In a two-phase mixture both the fluid and the solid each have a unique dielectric constant εᵣ. Each homogeneous mixture of the two will also have a unique value of εᵣ. The εᵣ value of the mixture will range from the εᵣ value of the fluid when the mixture is 100% fluid to the εᵣ value of the solid when the mixture is 100% solid. Once you have a unique value of εᵣ that correlates to each two-phase mixture, then by determining the εᵣ value you can determine the solid content of the mixture.

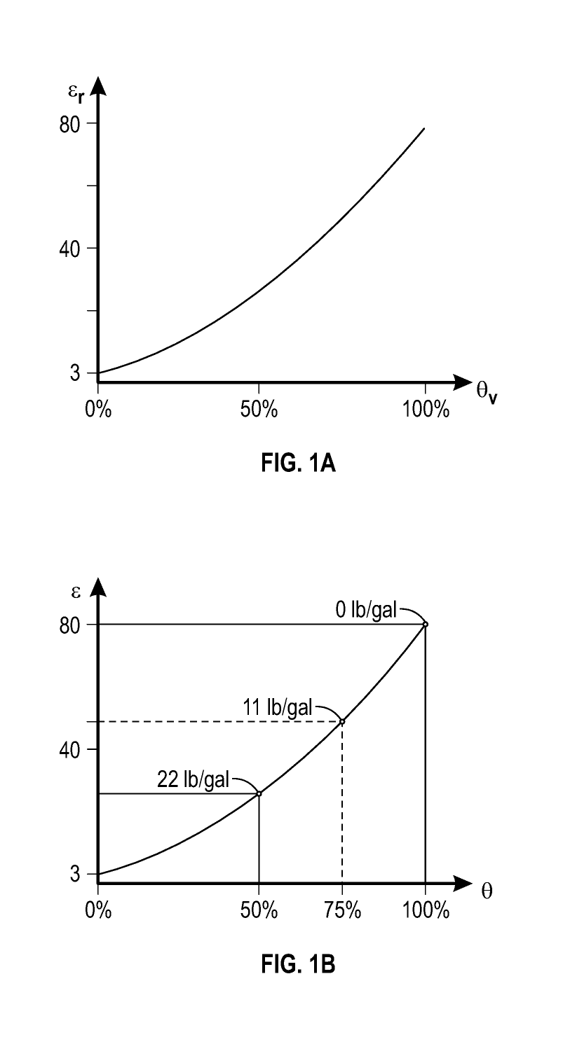

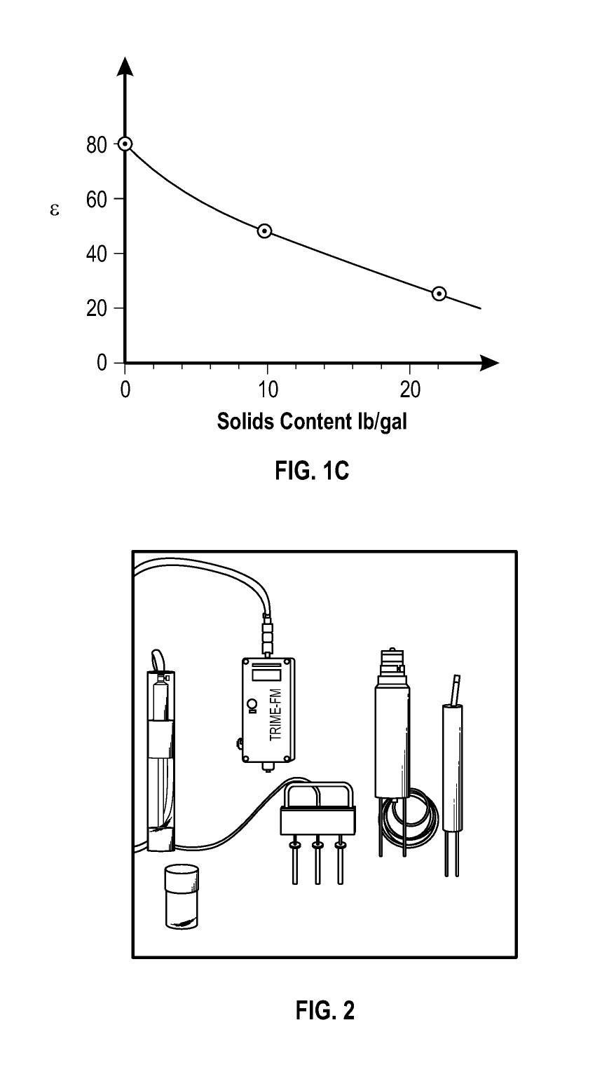

To illustrate, FIG. 1A shows a graph of εᵣ versus volumetric water content for a typical soil. Assume that this typical soil represents a fracturing fluid slurry containing a fracturing proppant having a dielectric constant of 3 (0% water on the chart). The chart is populated with εᵣ values ranging from 3 when the water content is 0% to a εᵣ value of 80 when the water content is 100%. For solids content we know that at 100% water there is 0 lb/gal sand concentration. By formulating two differing slurries, one at 75% water and another at 50%, we can add two data points to the known data. In this illustrative example we determine that at 75% water there is 11 lb/gal sand concentration and at 50% water there is 22 lb/gal sand concentration. From the modified graph shown as FIG. 1B we can determine a εᵣ value of 49 when the water content is 75%, which corresponds to the 11 lb/gal sand concentration. We further determine a εᵣ value of 28 when the water content is 50%, which corresponds to the 22 lb/gal sand concentration. We can further read a εᵣ value of 80 when the water content is 100% which corresponds to a 0 lb/gal sand concentration. Therefore the range of εᵣ values from 28 to 80 can be uniquely mapped to sand concentrations ranging from 22 lb/gal to 0 lb/gal as shown in Table 1.

TABLE 1

| | | Solids |
|---|---|---|
| Dielectric Constant | Water Content % | Concentration lb/gal |
| 28 | 50 | 22 |
| 49 | 75 | 11 |
| 80 | 100 | 0 |

By correlating the dielectric constant value versus the solids concentration from Table 1 data we obtain the graph as shown in FIG. 1C. Having developed this correlation we can now measure the dielectric constant of a flowing slurry stream of this particular fluid-solid mixture. Knowing the relationship as shown in FIG. 1C we can determine what the solids content of the slurry is at any εᵣ value that is measured, thereby giving an almost instantaneous determination of solids content.

In an embodiment of the present invention there is the desire to determine the solids content of a two-phase (solid-liquid) slurry of known solid material and carrier fluid in a flowing state. Ranges of fluid/solid mixtures are formulated and at known formulations (% fluid content and lb/gal solid content) the εᵣ value is measured and recorded. The solids content and the εᵣ values are graphed as εᵣ value versus % fluid content. The solids content for the mixture is determined for various known fluid contents, such as 0 lb/gal solid content at 100% fluid content. Knowing the solids content and the εᵣ value at the various fluid contents, the correlation between a mixtures εᵣ value and the solids content of the slurry is determined, the εᵣ value is then related to the percent solids or solids concentration. Therefore having determined this correlation, we can measure the εᵣ value of the flowing slurry in question and can then correlate the εᵣ value to the percent solids or solids concentration.

There are currently numerous commercial offerings of devices that can be used to determine a εᵣ value of a material. A few will be discussed below, but these are not an exhaustive listing of means to measure the εᵣ value of a material or mixture and are not to be seen as a critical dimension of the present invention. These devices and measurement means can be modified to achieve a system for a particular application.

Time Domain Reflectometry

In an embodiment the εᵣ value of a slurry is determined using Time Domain Reflectometry (TDR). TDR can determine the εᵣ value of a material by use of wave propagation. The TDR method is a transmission line technique and determines an apparent TDR permittivity from the travel time of an electromagnetic wave that propagates along a transmission line, usually two or more parallel metal rods embedded in a soil or sediment. TDR probes are usually between 10 and 30 cm in length and connected to the TDR via a coaxial cable.

One commercial soil probe is referred to as TRIME® (Time domain Reflectometry with Intelligent MicroElements. The TRIME soil probes are based on the TDR technique and developed to measure the dielectric constant of a material. A variety of TRIME TDR probes are shown in FIG. 2. The metal rods, stripes or plates are used as wave guides for the transmission of the TDR signal. The device generates a high frequency pulse which propagates along the wave guides generating an electromagnetic field around the probe. At the end of the wave guides, the pulse is reflected back to its source. The resulting transit time and dielectric constant are dependent on the moisture content of the material. This is represented by the equation below where l is transit length, t is transit time, c₀ is the speed of light in a vacuum, and εᵣ is the dielectric constant.

$${ɛ}_{r} = {( \frac{t * {c}_{0}}{l} )}^{2} \qquad Equation 1$$

There are a variety of probe arrangements and geometries in commercial use.

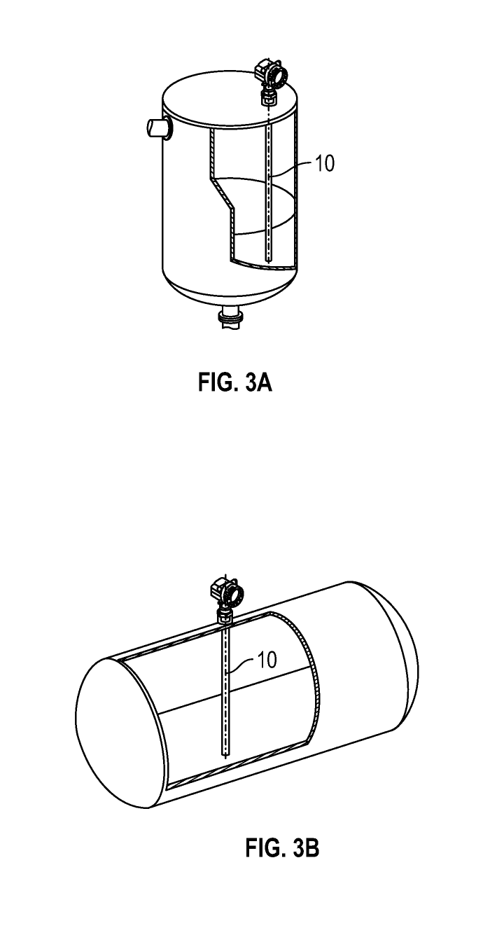

An alternate class of sensors uses TDR to determine the interface of a liquid and a gas. There is a significant commercial market for radar-based level sensors and many product offerings. An electromagnetic pulse is sent through air (Through-Air Radar, TAR) or guided with a rod or cable (Guided-Wave Radar, GWR) from a transmitter to a target. When the dielectric constant of the medium in the transmission path changes a reflection is generated. The time it takes for a reflection to return to the source is related to the distance to the discontinuity. Knowing that distance and the probe length (or tank height), fluid level can be determined. FIG. 3 is an illustration of the typical use of this sensor 10 in tanks of differing orientation. For the purposes of the present invention a GWR sensor can be placed with the wave-guide rod fully immersed in the slurry under investigation. The time it takes for a pulse to reach the end of the rod and return is dependent on the dielectric constant and thus the solids concentration of the slurry as previously discussed. The sensor would be used simply to obtain transit time.

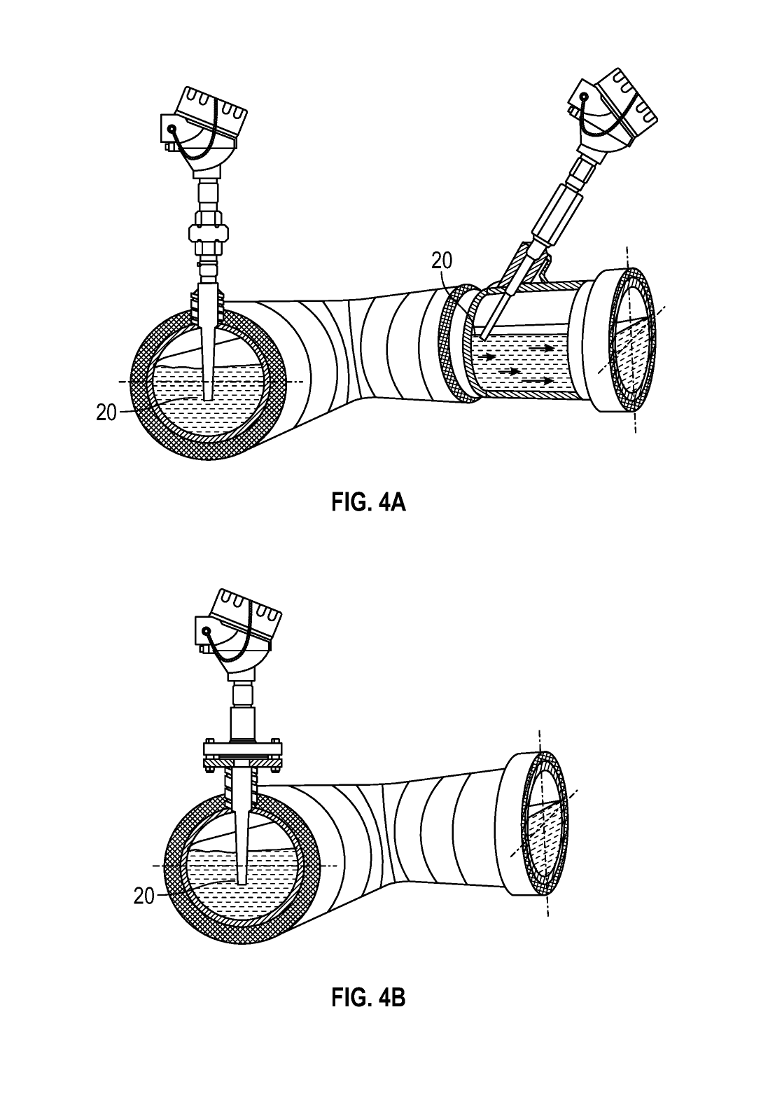

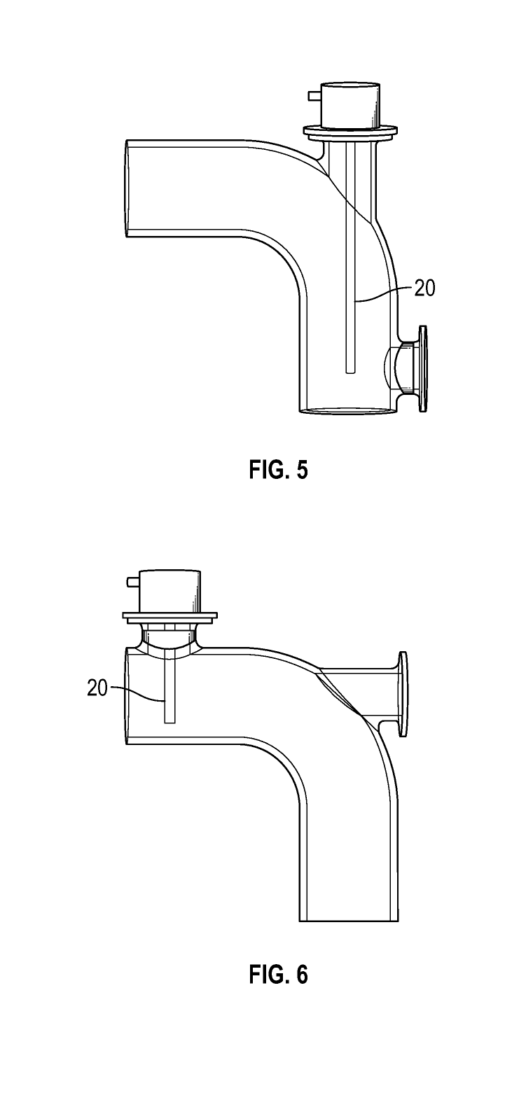

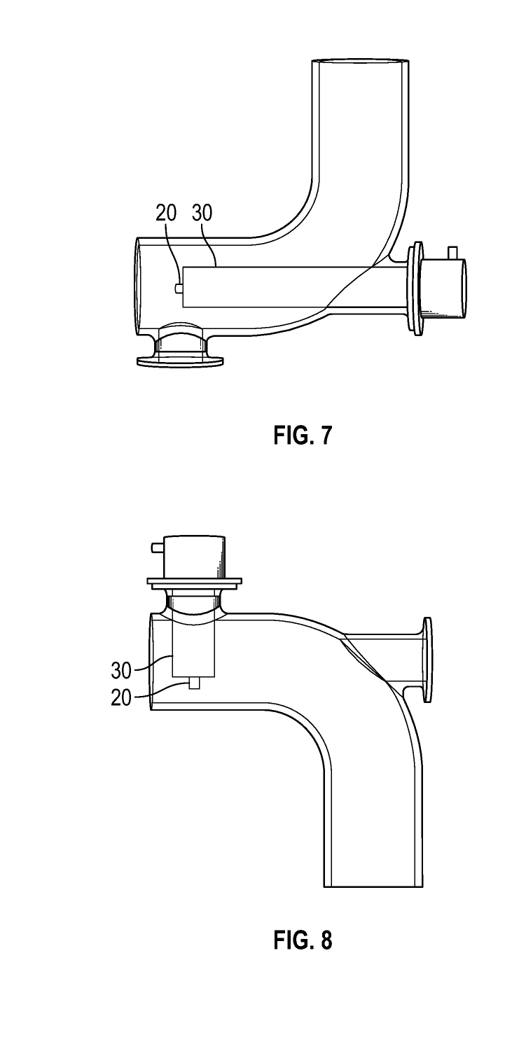

In an embodiment TDR is used to determine the dielectric constant and thus the moisture and solids content of a slurry. The probe can be a variation of commercial soil sensors or GWR level sensors. The probe can have a perforated outer pipe or tube for erosion protection. In practice a variation of commercial soil sensors or GWR level sensors could be used. For example a probe can be placed directly in a Frac blender tub to measure the dielectric constant of a fracturing fluid prior to it being pumped downhole. Alternately a probe can be placed directly in a cement mix tub to measure the dielectric constant of a cement slurry. FIGS. 4 through 8 illustrate various non-limiting embodiments of probe 20 locations. Embodiments shown include probe positions in parallel and perpendicular locations in relation to the flow stream, although installations in locations other than parallel and perpendicular can also be used. FIGS. 7 and 8 illustrate embodiments having a protective tube 30 for erosion protection for the measurement probe.

Capacitance

In an embodiment the dielectric constant is determined by forming a capacitor with the dielectric material (the slurry). The resulting capacitance value (or a representative value) is determined through one of several means. This capacitance value is a function of the dielectric constant and therefore the dielectric constant can be determined. The geometry of the capacitor can be, but is not limited to, coaxial, parallel plate, stacked parallel plates, curved plates, parallel wires, or spherical.

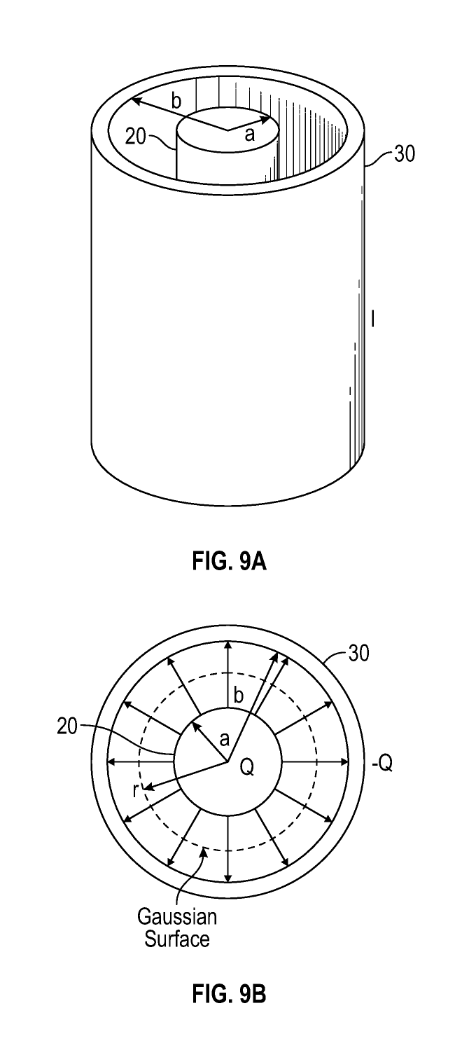

To illustrate, a coaxial capacitor can be fabricated with a centered rod and an outer cylinder as conductors. FIG. 9 illustrates this embodiment. The slurry flows through the void and becomes the dielectric material of the capacitor. The capacitance of the construction is described by the equations below where b is the ID of the outer cylinder and a is the OD of the inner cylinder (the rod) and L is the length of the probe.

$$C = \frac{2 π L {ɛ}_{r}}{ln ( b / a )} \qquad Equation 2$$

Rearranging this equation the dielectric constant can be solved for.

$${ɛ}_{r} = \frac{C ln ( b / a )}{2 π L} \qquad Equation 3$$

Thus by determining the capacitance of the construction the dielectric constant can be obtained which can then be correlated to solids concentration.

In an embodiment a capacitance watercut meter or a variation thereof is used. These typically have coaxial geometries. An embodiment consists of a pipe (the outer cylinder) and an insulated rod (the inner cylinder) wherein the oil/water mixture provides the primary dielectric material of the capacitor. The capacitance of the slurry is measured and as the capacitance value is a function of the dielectric constant, the dielectric constant can be determined. The εᵣ value is then related to the percent solids or sand concentration as discussed previously.

In an embodiment the capacitance measurement are capacitive level sensors or a variation thereof. These are available commercially from a variety of vendors. They can be continuous level sensors with a long rod or a point-level sensor to detect the presence/absence of a material. These are essentially coaxial-like capacitors where the probe rod is one conductor and a pipe, tub, tank, or bin as the other conductor. The material in the container provides the dielectric. The coaxial capacitance equation has a length term as well as the dielectric constant term. For continuous level measurement, εᵣ must be known and fixed. Thus the response of the instrument is affected by the level of the material being measured (the “length” of material on the probe). In an embodiment the length is a fixed value through constant immersion so that the instruments output would respond to εᵣ.

If a commercial watercut meter or capacitance level sensor is used as the basis for an embodiment, that sensor will likely have the electronic circuitry needed to get an output related to εᵣ. This output may have to be read by a data acquisition computer where additional algorithms are applied to convert εᵣ to sand concentration or percent solids. If a capacitance sensor is fabricated, the capacitance can be determined in a variety of ways including, but not limited to, the following methods.

An oscillator can be made where the capacitance sensor is a component setting the frequency. The frequency is then counted and related to capacitance and/or εᵣ.

An oscillating ramp generator can be made where voltage ramp time is dependent on the capacitance sensor. The ramp time can be measured with a counter, or the ramp can be processed to provide an analog indication of capacitance.

The complex impedance can be measured much like a commercial LCR meter. In this method, the capacitor is excited with an AC voltage that is monitored with the current.

The amplitude and phase relationship can be used to determine impedance, including capacitance. As the capacitance value is a function of the dielectric constant, the dielectric constant can be determined. The εᵣ value is then related to the percent solids or sand concentration as discussed previously.

A capacitance bridge can be made which compares the capacitance sensor to known capacitance values through a bridge arrangement. While this can be quite precise, field implementation may be difficult when dealing with flowing slurry material.

The fabrication of an ideal capacitor of some geometry, e.g. coaxial, may be difficult. Thus, textbook equations will likely have to be supplemented with empirical adjustments. Variations in piping may require calibration with a particular flow tube.

Although the invention is primarily directed toward the measurement of sand concentration of fracturing fluid slurries, the methods disclosed herein can be used to determine the solids concentration of any particulate suspended in any type of fluid. The type of solid and the type of fluid is not limiting to the method disclosed herein. Examples include any particulate laden fluid such as cementing fluids, spacer fluids, gravel pack fluids, coal slurries, etc. The particulate material can include dry chemicals suspended in a fluid. Optionally the solid material can consist of various solid additives such as bits of plastic, etc. Although the invention has been described primarily as a method of determining the solids content of a slurry, it can also be used in determining the fluid content of the slurry.

In an embodiment the slurry being investigated is a solid & liquid slurry. Optional embodiments can include, as non-limiting examples, where the slurry is a suspension, solution, colloid or other forms of slurries.

The various embodiments of the present invention can be joined in combination with other embodiments of the invention and the listed embodiments herein are not meant to limit the invention. All combinations of various embodiments of the invention are enabled, even if not given in a particular example herein.

While illustrative embodiments have been depicted and described, modifications thereof can be made by one skilled in the art without departing from the scope of the disclosure. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

While compositions and methods are described in terms of “comprising,” “containing,” or “including” various components or steps, the compositions and methods can also “consist essentially of” or “consist of” the various components and steps. Also, the terms in the claims have their plain, ordinary meaning unless otherwise explicitly and clearly defined by the patentee.

1. A method for determining the composition of a two-phase slurry, comprising:
determining the dielectric constant of a slurry at various known compositions;
correlating the dielectric constant of the slurry with the composition of the slurry at the known compositions;
determining the dielectric constant of an unknown slurry composition; and
calculating the slurry composition based on the relationship between the dielectric constant and the composition of the slurry.

2. The method of claim 1, wherein determining the composition of the two-phase slurry includes determining the solids content of the two-phase slurry.

3. The method of claim 1, wherein determining the composition of the two-phase slurry includes determining the fluid content of the two-phase slurry.

4. The method of claim 1, wherein the two-phase slurry is a fracturing fluid containing proppant material used to stimulate a subterranean formation.

5. The method of claim 1, wherein the two-phase slurry is cement slurry.

6. The method of claim 1, wherein the dielectric constant of the unknown slurry is obtained using TDR (Time Domain Reflectometry).

7. The method of claim 1, wherein the dielectric constant of the unknown slurry is obtained by measuring the capacitance of the slurry.

8. A method for determining the solids content of a two-phase (solid-liquid) slurry comprising a known solid and a known carrier fluid, comprising:
preparing slurry samples of known solids content at various compositions;
measuring the dielectric constant of the slurry at said various known compositions;
correlating the dielectric constant with the fluid content of the slurry samples;
correlating the solids content of the slurry samples with the fluid content of the slurry samples;
correlating the dielectric constant with the solids content of the slurry samples;
measuring the dielectric constant of an unknown slurry composition; and
calculating the solids content of the unknown slurry composition based on the relationship between the dielectric constant and the solids content of the slurry.

9. The method of claim 8, wherein the two-phase slurry is sand & water slurry.

10. The method of claim 8, wherein the two-phase slurry is a fracturing fluid containing proppant material used to stimulate a subterranean formation.

11. The method of claim 8, wherein the two-phase slurry is cement slurry.

12. The method of claim 8, wherein the dielectric constant of the unknown slurry is obtained using TDR (Time Domain Reflectometry).

13. The method of claim 8, wherein the dielectric constant of the unknown slurry is obtained by measuring the capacitance of the slurry.

14. A method for determining the solids content of a two-phase slurry, comprising:
determining the dielectric constant of the solid phase;
determining the dielectric constant of the liquid phase;
determining the dielectric constant of an unknown slurry composition;
calculating the solids content based on the relationship between the slurry dielectric constant and the solids content of the slurry using an appropriate algorithm with the dielectric constant of the solid phase and the dielectric constant of the liquid phase.

15. The method of claim 14, further comprising calculating the fluid content of the two-phase slurry based on the relationship between the slurry dielectric constant and the fluid content of the slurry using an appropriate algorithm with the dielectric constant of the solid phase and the dielectric constant of the liquid phase.

16. The method of claim 14, wherein the two-phase slurry is sand & water slurry.

17. The method of claim 14, wherein the two-phase slurry is a fracturing fluid containing proppant material used to stimulate a subterranean formation.

18. The method of claim 14, wherein the two-phase slurry is cement slurry.

19. The method of claim 14, wherein the dielectric constant of the unknown slurry is obtained by at least one of using TDR (Time Domain Reflectometry) and measuring the capacitance of the slurry.

20. (canceled)

21. (canceled)

22. The method of claim 1, further comprising calculating a fluid content of the two-phase slurry based on the relationship between the dielectric constant and the fluid content of the slurry.
